(19) United States
(12) Patent Application Publication
(10) Pub. No.: US 2004/0106965 A1
(43) Pub. Date: Jun. 3, 2004

(54) Methods and apparatus for treatment of degenerative retinal disease via indirect electrical stimulation

(76) Inventor: Alan Y. Chow, Wheaton, IL (US)

Correspondence Address: GENERAL NUMBER 00757, BRINKS HOFER GILSON & LIONE, P.O. BOX 10395, CHICAGO, IL, 60611, US

(21) Appl. No.: 10/606,117

(22) Filed: Jun. 24, 2003

Related U.S. Application Data
(63) Continuation-in-part of application No. 10/056,793, filed on Jan. 23, 2002.
(60) Provisional application No. 60/301,877, filed on Jun. 29, 2001.

(51) Int. Cl. A61N 1/18

(52) U.S. Cl. 607/54

(57) ABSTRACT

To provide indirect electrical stimulation for treatment of degenerative retinal diseases, such stimulation is applied to surface structures of the eye. A source of an electrical stimulation signal is coupled to at least one stimulating electrode configured for chronic contact with a surface structure of an eyeball. Additionally, at least one return electrode, which may be configured either for contact with conductive biological tissue distant from the eyeball or for contact with a surface structure of the eyeball, is also coupled to the source. The source of the electrical stimulation signal may be implemented internal to a body of a patient, external to the body or through a combined internal/external approach. The active and return electrodes are preferably arranged such that the circuit created by the source, stimulating electrode, biological tissue and return electrode provides trans-retinal electrical stimulation to thereby effect treatment.

### CROSS-REFERENCE TO RELATED APPLICATIONS

The instant application is a continuation-in-part of prior U.S. patent application Ser. No. 10/056,793, entitled “METHODS FOR IMPROVING DAMAGED RETINAL CELL FUNCTION”, filed Jan. 23, 2002, which prior application claims the benefit of Provisional U.S. Patent Application Serial No. 60/301,877, entitled “METHOD OF IMPLANTING A RETINAL STIMULATION DEVICE FOR GENERALIZED RETINAL ELECTRICAL STIMULATION”, filed Jun. 29, 2001, the entirety of which are incorporated by reference herein.

### FIELD OF THE INVENTION

The present invention relates to treatment of degenerative retinal disease and, in particular, to methods and apparatus for treatment thereof based on external electrical stimulation.

### BACKGROUND

Many human retinal diseases cause vision loss by partial to complete destruction of the vascular layers of the eye that include the choroid and choriocapillaris, both of which nourish the outer anatomical retina and a portion of the inner anatomical retina of the eye.

Many other retinal diseases cause vision loss due to partial to complete degeneration of one or both of the two anatomical retinal layers directly, due to inherent abnormalities of these layers. The components of the retinal layers include Bruch's membrane and retinal pigment epithelium which comprise the “outer anatomical retinal layer”, and the photoreceptor, outer nuclear, outer plexiform, inner nuclear, inner plexiform, amacrine cell, ganglion cell and nerve fiber layers which comprise the “inner anatomical retinal layer”, also known as the “neuroretina”. The outer portion of the neuroretina is comprised of the photoreceptor and bipolar cell layers and is also known as the “outer retina” which is to be distinguished from the “outer anatomical retinal layer” as defined above. Loss of function of the outer retina is commonly the result of dysfunction of the outer anatomical retinal layer that provides nourishment to the outer retina and/or to direct defects of the outer retina itself. The final common result, however, is dysfunction of the outer retina that contains the light sensing cells, the photoreceptors. Some of these “outer retina” diseases include age-related macula degeneration, retinitis pigmentosa, choroidal disease, long-term retinal detachment, diabetic retinopathies, Stargardt's disease, choroideremia, Best's disease, and rupture of the choroid. The inner portion of the neuroretina, however, often remains functionally and anatomically quite intact and may be activated by the appropriate stimuli.

While researchers have reported efforts to restore visual function in humans by transplanting a variety of retinal cells and retinal layers from donors to the subretinal space of recipients, no sustained visual improvement in such recipients has been widely accepted by the medical community.

Multiple methods and devices to produce prosthetic artificial vision based on patterned electrical stimulation of the neuroretina in contact with, or in close proximity to, the source of electrical stimulation are known. These devices typically employ arrays of stimulating electrodes powered by photodiodes or microphotodiodes disposed on the epiretinal side (the surface of the retina facing the vitreous cavity) or the subretinal side (the underneath side) of the neuroretina. For example, Chow et al. have described various designs for implants to be inserted in the sub-retinal space, i.e., a space created between the inner and outer retinal layers, in U.S. Pat. Nos. 5,016,633; 5,024,223; 5,397,350; 5,556,423; 5,895,415; 6,230,057; 6,389,317 and 6,427,087. Generally, the implants described in these patents are placed in contact with the photoreceptor layer of the inner retina such that electrodes on the implants can provide stimulating currents, derived from the photovoltaic conversion of incident light, to the inner retina. Additionally, techniques and devices for inserting such implants into the sub-retinal space are also described in various ones of these patents, e.g., U.S. Pat. Nos. 5,016,633; 5,024,223 and 6,389,317.

Cellular electrical signals also play important developmental roles, enabling nerve cells to develop and function properly. For example, nerve cells undergo constant remodeling, or “arborization”, during development related to electric signaling. First an extensive preliminary network is formed that is then “pruned” and refined by mechanisms that include cell death, selective growth, loss of neurites (axonal and dendritic outgrowths), and the stabilization and elimination of synapses (Neely and Nicholls, 1995). If a neuron fails to exhibit or is inhibited from transducing normal electrical activity during arborization, axons fail to retract branches that had grown to inappropriate positions.

The application of electric currents to organ systems other than the eye is known to promote and maintain certain cellular functions, including bone growth, spinal cord growth and cochlear spiral ganglion cell preservation (Acheson et al., 1991; Dooley et al., 1978; Evans et al., 2001; Kane, 1988; Koyama et al., 1997; Lagey et al., 1986; Leake et al., 1991; Leake et al., 1999; Politis and Zanakis, 1988a; Politis and Zanakis, 1988b; Politis and Zanakis, 1989; Politis et al., 1988a; Politis et al., 1988b).

In other studies, the application of growth and neurotrophic-type factors was found to promote and maintain certain retinal cellular functions. For example, brain-derived neurotrophic factor (BDNF), neurotrophin-4 (NT-4), neurotrophin-5 (NT-5), fibroblastic growth factor (FGF) and glial cell line-derived neurotrophic factor (GDNF) have been shown to enhanced neurite outgrowth of retinal ganglion cells and to increase their survival in cell culture. GDNF has been shown to preserve rod photoreceptors in the rd/rd mouse, an animal model of retinal degeneration. Nerve growth factor (NGF) injected into the intra-ocular area of the C3H mouse, also a model of retinal degeneration, results in a significant increase of surviving photoreceptor cells compared to controls (Bosco and Linden, 1999; Caleo et al., 1999; Carmignoto et al., 1989; Cui et al., 1998; Frasson et al., 1999; Lambiase and Aloe, 1996; Reh et al., 1996). No methods or devices, however, to improve the general inherent visual function of damaged retinal cells distant from a source of electrical stimulation through the use of chronic electrical stimulation applied to the neuroretina from either within the eye or indirectly via contact with surface structures of the eye are known.

### BRIEF SUMMARY

The present invention provides techniques for preventive or therapeutic treatment of degenerative retinal disease through the application of electrical stimulation. In particular, the present invention concerns the use of indirect electrical stimulation for such treatment. Generally, this is achieved with a device comprising a source of an electrical stimulation signal coupled to at least one stimulating electrode configured for chronic contact with a surface structure of an eyeball and at least one return electrode, which at least one return electrode may be configured either for contact with conductive biological tissue distant from the eyeball or for contact with a surface structure of the eyeball. Surface structures of the eyeball may be categorized as either external surface structures (e.g., conjunctiva and cornea) or internal surface structures (e.g., sclera, episclera, intramuscular septum, Tenon's capsule, etc.). The source of the electrical stimulation signal may be implemented internal to a body of a patient, external to the body or through a combined internal/external approach. In one embodiment of the present invention, a plurality of stimulating electrodes is arranged in a ring formation. The at least one return electrode may also be arranged in the ring formation, particularly in an interleaved fashion with the stimulating electrodes. The active and return electrodes are preferably arranged such that the circuit created by the source, stimulating electrode, biological tissue and return electrode provides trans-retinal electrical stimulation to thereby effect treatment.

### BRIEF DESCRIPTION OF THE DRAWINGS

FIG. 1 is a cross-sectional top view of a human eye.

FIG. 2 is a cross-section through a human eye indicating layers of the outer and inner anatomical retina, as indicated by the inset of FIG. 1.

FIG. 3 is a cross-sectional view showing the placement of a first embodiment of a RSD in the subretinal space of the eye.

FIG. 4 is a cross-sectional view showing the placement of a second embodiment of a RSD in the subretinal space of an eye with a silicon tail and ground return electrode in the vitreous cavity.

FIG. 5 is a cross-sectional view of a modified second embodiment of FIG. 4 showing the main photodiode portion of the RSD in the subretinal space and the extended tail of the RSD in the anterior chamber of the eye where it terminates in a photodiode array connected in series and/or parallel with the main photodiode of the RSD to provide additional voltage and/or current to stimulate the retina. In this latter device the ground return electrode is located on the photodiode array placed in the eye's anterior chamber.

FIG. 6 is a cross-sectional view showing the placement of a third embodiment of a RSD implanted on an epiretinal surface of the retina and secured to the retina by tacks.

FIG. 7 is a schematic block diagram of a prior art technique for indirect electrical stimulation.

FIG. 8 is a schematic block diagram of another prior art technique for indirect electrical stimulation.

FIG. 9 is a schematic block diagram of yet another prior art technique for indirect electrical stimulation.

FIG. 10 is a cross-sectional view showing placement of a third embodiment RSD implanted between a conjunctiva and a scleral surface as a first technique for indirect electrical stimulation in accordance with the present invention.

FIG. 11 is a schematic block diagram of a second technique for indirect electrical stimulation in accordance with the present invention.

FIG. 12 is a schematic block diagram of a third technique for indirect electrical stimulation in accordance with the present invention.

FIG. 13 is a partial cross-sectional side view of a human eye and surrounding structures.

FIG. 14 is a partial cross-sectional magnified view of a region of the eye illustrated in FIG. 13.

FIG. 15 is a side view of a human eye illustrating application of a corneal electrode in accordance with an embodiment of the present invention.

FIG. 16 is a side view of a human eye illustrating application of an epi-conjunctival electrode in accordance with an embodiment of the present invention.

FIG. 17 is a side view of a human eye illustrating application of a fiber electrode in a conjunctival formix in accordance with an embodiment of the present invention.

FIG. 18 is a side view of a human eye illustrating application of a plurality of electrode arrays applied to an internal surface structure in accordance with an embodiment of the present invention.

FIG. 19 is a side view of a human eye illustrating application of an electrode array to an internal surface structure in accordance with an embodiment of the present invention.

### DETAILED DESCRIPTION OF THE PRESENTLY PREFERRED EMBODIMENTS

In the course of testing for the safety and efficacy of retinal implants in humans blinded by retinitis pigmentosa, an unexpected and surprising observation was made: even though the implants were placed at a discrete location in the subretinal space (acting as a prosthesis), vision was improved not only in those discrete locations as expected, but also in distant locations of the retina. Thus chronic electrical stimulation in specific locations enhanced retinal cell function throughout the eye. This “halo effect” can be used to improve vision in those individuals who suffer from diseases, conditions and traumas that have damaged the outer retinal layer but leave the inner retinal layer at least partially intact. Although prosthetic electrical devices designed to replace damaged or missing retinal cells have been used to treat vision loss caused by outer retinal degeneration, electrical stimulation to improve large areas of retinal cell visual function is novel. As a non-limiting explanation, the promotion of improved retinal cell visual function by chronic electrical stimulation may be explained by the stimulation of production and release of growth factors; more specifically, neurotrophic-type growth factors, by the stimulated retinas. The synthesis and/or secretion of neurotrophic factors would then improve retinal cell function and survival in conditions where these activities would be lost.

Accordingly, the present invention discloses both novel devices and methods to electrically stimulate the retina to improve large areas of retinal visual function and to protect the retina from degeneration. As described in greater detail below, the devices and methods disclosed herein may be generally categorized as direct and indirect. Direct techniques involve stimulation of a retina wherein the stimulus traverses substantially no intervening biological structures. Conversely, indirect techniques encompass stimulation of a retina wherein the stimulus must traverse one or more intervening biological structures.

Subject/Patient

A subject (patient) may be a human being or a non-human animal, but is preferably a human. Usually the individual has suffered some type of retinal damage and/or degeneration that results in some degree of visual loss and/or has a condition that will result in retinal damage and/or degeneration. A normal (healthy) subject does not have a condition that will result in retinal damage and/or degeneration and/or has not suffered retinal damage and/or degeneration.

Improving visual function

Improving visual function refers to improving a targeted function of the eye, selected by the artisan, and includes improving any to all of the following capabilities of the eye, retina and visual system: perception of brightness in the presence of light, perception of darkness in the absence of light, perceptions of contrast, color, shape, resolution, movement and visual field size.

Primary visual degradation means loss of visual function due to malfunctioning, damaged or degeneration of structures found in the eye. Secondary visual degradation means loss of visual function due to secondary damage, typically from lack of use of the vision-associated portions of the brain. Improving visual function means to improve the visual function of primary visual degradation, secondary visual degradation or both.

Eye/Eyeball

The eye (or eyeball) has the usual definition in the art. Eye includes all interior and exterior surfaces, components, contents and cavities of the eye. The eye does not include the eyelid or optic nerve.

The retina of the eye can be divided into sectors as is commonly accepted in the art. Such sectors are described by the use of the terms temporal, nasal, superior, inferior, by clock hour designation, and by the number of degrees away from the macula. For example, the temporal sector of the retina is the retina temporal to a perpendicular plane cutting through retina from the 12 o'clock to the 6 o'clock positions and through the macula. In another example, the superior sector is the retina superior to a perpendicular plane cutting through the 9 o'clock to 3 o'clock positions and through the macula. In a further example, the superior-temporal sector is the intersection of these two sectors, a pie-shaped area delineated from the 9 o'clock position of the peripheral retina to the macula and then clockwise to the 12 o'clock position. More specific locations of the retina can be designated by degrees away from the macula and clock hour location: for example, 20 degrees away from the macula at the 3 o'clock (nasal) position. The number of degrees away from the macula is in visual axes degrees. These axes all intersect through the lens of the eye.

The visual field sectors correspond oppositely to the retinal sectors as is commonly understood in the art. For example, the superior-temporal sector of the retina corresponds to the inferior-nasal portion of the visual field.

Peripheral

To be peripheral to an object, device or other landmark includes all surrounding parts, but not the object, device or landmark, i.e., the object, device or landmark, together with the peripheral portion, constitutes the whole.

Light

Light refers not only to the electromagnetic spectrum that humans can readily perceive visually (approximately 400 nm to 750 nm), but also includes ultraviolet light (<400 nm in wavelength) as well as infrared light (>750 nm in wavelength).

Indications

The invention can be used to improve visual function in subjects in which the retina is damaged by disease, degeneration, condition, or trauma and/or to slow down or stop the progression of damage by disease, degeneration, condition or trauma. Common diseases, conditions, degeneration or trauma that are particularly amenable to this treatment include age-related macula degeneration, retinitis pigmentosa, Leber's congenital amaurosis, Stargardt's disease, Best's disease, diabetic retinopathy, long-term retinal detachment, and choroidal damage.

Eye Structure

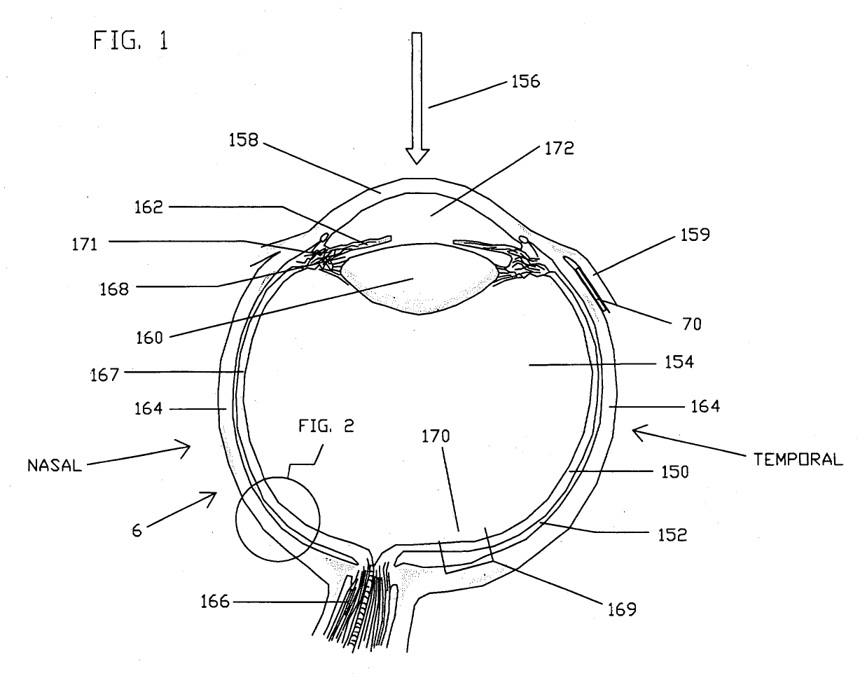

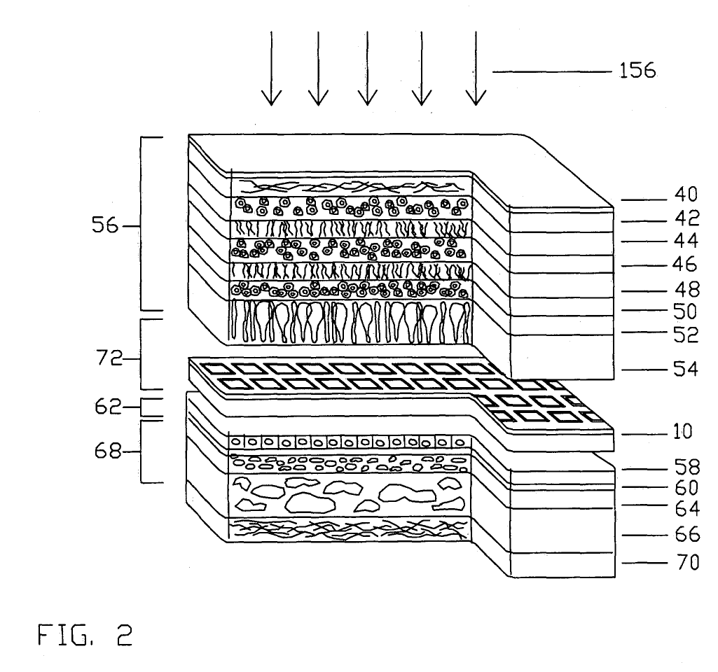

Referring to the drawings, FIG. 1 illustrates a section through the eyeball. The neuroretina 150 comprises multiple layers of cells and structures (see FIG. 2). The photoreceptor components of the retina are situated within the neuroretina which covers the internal posterior cavity of the eye, terminating anteriorly at the ora serrata 167. The ciliary body 168 and the iris 162 are covered by extensions of the retina, lacking photoreceptor components. The outermost layers of the eye consist of the sclera 164 and cornea 158. The sclera is pierced by the emerging optic nerve 166. The lens 160 and vitreous cavity 154 are also indicated. The macula 169 of the retina is typically a 3 mm by 5 mm oval region, at the center of which is the fovea 170.

The layers of the eye at the posterior pole from inside to outside are shown in FIG. 2: internal limiting membrane 40, nerve fiber layer 42, ganglion and amacrine cell layer 44, inner plexiform 46, inner nuclear layer 48, outer plexiform 50, outer nuclear and bipolar cell layer 52, and photoreceptor layer 54, all of which constitute the anatomical inner retinal layer, also known as the neuroretina 56. The retinal pigment epithelium 58 and Bruch's membrane 60 constitute the outer retinal layer 62. The choriocapillaris 64 and choroid 66 comprise the choroidal vasculature 68. The outer coat of the eye is the sclera 70. Light 156 enters the retina as shown.

Direct Stimulation

Any device that provides (or can apply) electrical stimulation, diffuse or discrete, to the eye can be used as a source of electrical stimulation. Preferably, these devices are retina stimulation devices (RSDs); more preferably, the RSDs are powered by incident light, ambient and/or amplified, although other means, such as batteries, external solar cells, supplied electrical current or potential voltage, may also be used. Such external power may be provided to the RSDs via direct electrical conductor and/or by electromagnetic power such as but not limited to radio frequency signals and light. The RSDs may be supplied such external power in a pattern such as cyclically, and/or in complex waveform patterns. Such external power provided to the RSDs may also be activated and deactivated by a user at will, which may be desirable when a user is sleeping. One or a plurality of devices may be used to apply electrical stimulation.

A variety of electrical devices have been described (Chow, U.S. Pat. No. 5,024,223,1991; Chow and Chow, U.S. Pat. No. 5,397,350, 1995; Chow and Chow, U.S. Pat. No. 5,556,423, 1996; Chow and Chow, 1997; Chow et al., 2001; Chow and Peachey, 1999; Chow and Chow, U.S. Pat. No. 5,895,415,1999; Chow and Chow, U.S. Pat. No. 6,230,057 B1, 2001), and are hereby incorporated by reference.

The RSD may comprise a disk-shaped silicon chip device, approximately 2 mm in diameter and 25 μm in thickness, comprising one or more groups of one or more photodiodes having one or more stimulating electrodes and one or more ground return electrodes. The RSD can be flexible or rigid and may be designed to conform to the structural curvature of the outside or inside of the eye, the subretinal space, the epiretinal surface, and/or the subscleral space. Also, the RSD may consist of multiple electrically isolated subunits connected by a flexible mesh. RSDs may be fabricated to function suitably with diameters that vary from 0.005 to 25 mm, and thicknesses that vary from 0.2 μm to 1000 μm, although those skilled in the art will appreciate that dimensions falling outside of the aforementioned values may also be suitable. The stimulating electrode or electrodes contacting the epiretinal or the subretinal side of the neuroretina may be the anode or cathode with the ground return electrode being the opposite polarity of the stimulating electrode. If the electrodes are on the eye surface, the stimulating electrode or electrodes contacting the outside of the eye may also be the anode or the cathode with the ground return electrode being the opposite polarity of the stimulating electrode.

In a first embodiment, the silicon chip RSD is a single photodiode 2 mm diameter and 25 μm thick with its photoactive surface facing incident light and its retinal stimulating electrode disposed on the same surface and electrically connected to the photodiode. On the opposite surface of the RSD is an electrode electrically connected to the photodiode that serves as the ground return electrode for the RSD. In use, the RSD silicon chip is preferably implanted surgically into the subretinal space of an eye in a paracentral location relative to the macula (i.e., peripheral to the macula). In this embodiment, it is preferred that the retinal stimulating electrode on the photoactive surface of the RSD photodiode is in contact with the inner retina from the subretinal space and is facing incident light, and the electrode is a cathode. Diffuse electric currents developed by the cathode, when the RSD is exposed to light, stimulate the neuroretina above, surrounding, and at a distance from the RSD to improve the damaged retina's inherent visual function. Such visual function improvement has been observed in a clinical study involving multiple patients implanted with such devices, resulting from chronic subretinal electrical stimulation produced by an implanted, high pixel density, artificial silicon retina device. However, it is recognized that a high pixel density of a retina stimulator is not necessary to achieve a general electrical stimulation of the retina. If needed, more than one RSD is implanted in an eye to stimulate a larger area of retina, and multiple RSDs would preferably be implanted in paracentral locations relative to the macula such as one in each of the four paracentral quadrants, approximately, but not limited to, 5 to 80 degrees peripheral to the macula.

In second embodiment, the electrical ground of the RSD is brought into the vitreous cavity via an insulated conductor preferably fabricated on a silicon tail that is part of the RSD with an exposed ground return electrode at the end of the conductor on the tail. This configuration directs the electrical current flow more efficiently between the stimulating and ground return electrodes of the RSD into a more through-the-retina, transretinal route and also through a smaller area of the neuroretina compared to the first RSD embodiment without this tail configuration. A modification of this second embodiment extends the tail into the lens capsule of the eye where it terminates in a photodiode array connected in series and/or parallel with the main RSD to provide additional voltage and/or current to stimulate the neuroretina. The purpose of placing the photodiode array in the lens capsule is to allow the photodiode array to be exposed to brighter intensities of incident light. In this modification of the preferred embodiment, the ground return electrode is located on the photodiode array placed in the lens capsule.

In yet another embodiment, at least two photodiodes are fabricated on the RSD that are electrically connected in series to produce higher voltages and higher resultant currents than is possible without such series connections. The RSD is fabricated in versions where the ground return electrode is located either in the subretinal space, or in the vitreous cavity at the end of a silicon tail (Chow and Chow, U.S. application Ser. No. 09/539,399).

In yet another embodiment, at least two photodiodes are fabricated on a RSD and electrically connected in a reverse parallel manner such as in an Opsistor fashion (Chow and Chow, U.S. Pat. No. 5,837,995, 1998) to provide biphasic and variable levels of stimulating electric currents both controlled by the use of different wavelengths of external visible and/or infrared light.

In yet another embodiment, fenestrations are fabricated into any of the aforementioned embodiments of the RSD. The fenestrations allow nourishment and oxygen to flow beneficially from the choroidal circulation and the outer anatomical retina into the inner anatomical retina for RSDs placed in the subretinal space.

With regard to FIG. 2, when an RSD 10 is inserted in the subretinal layer, it is inserted within the retina between the inner retinal layer 56 (that may or may not contain a functional photoreceptor layer 54) and the outer retinal layer 62, i.e., in the potential space zone. The overlying inner retinal layer consisting of photoreceptors and their cell bodies 54, 52, bipolar cells 48 and horizontal cells 52 are also shown. The bipolar cells 48 and ganglion cells 44 are in the innermost area of the inner retinal layer, processing visual cues such as electric signals for distant transmission through the optic nerve to the brain.

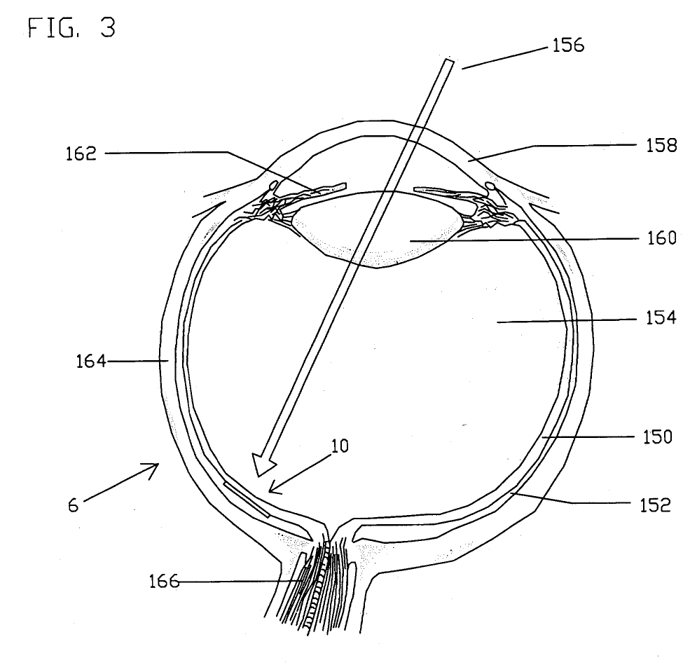

FIG. 3 is a cross-sectional view showing a first embodiment RSD 10 implanted in the eye 6 in the subretinal space between the neuroretina 150 and the retinal pigment epithelium 152. Light 156 entering the eye 6 through the cornea 158 and lens 160 is focused onto the RSD 10. Electrical current is generated by the RSD, which may comprise one or more photodiodes optionally connected in series or in reverse parallel fashion, and provides beneficial stimulation to the overlying neuroretina 150. For purposes of reference, other structures of the eye 6 that are shown are the iris 162, the sclera 164 and the optic nerve 166.

FIG. 4 is a cross-sectional view showing a second embodiment RSD 20 implanted in the eye 6. The stimulating electrode unit 23 is located in the subretinal space between the neuroretina 150 and the retinal pigment epithelium 152 while the ground return electrode unit 26 is located in the vitreous cavity 154. Light 156 entering the eye 6 through the cornea 158 and lens 160 is focused onto the RSD 20. Electrical current is generated by the RSD, which may comprise one or more photodiodes optionally connected in series or in reverse parallel fashion, which provides beneficial stimulation to the overlying and surrounding neuroretina 150. For purposes of reference, other structures of the eye 6 that are shown are the iris 162, the sclera 164, the optic nerve 166, lens 160 and cornea 158.

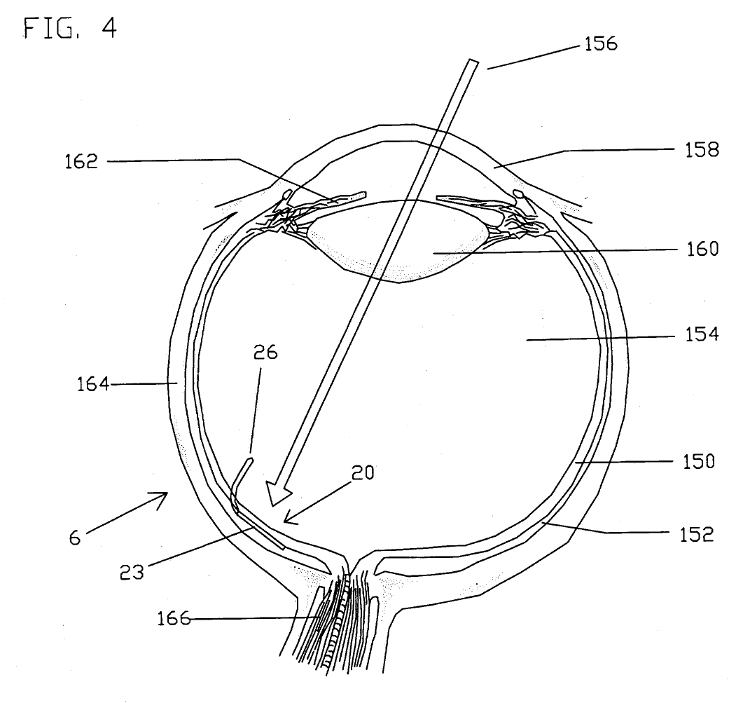

FIG. 5 shows a cross-sectional view of a modification 20e of the second embodiment RSD of FIG. 4 that includes an attached tail extension 27 that electrically connects with at least one bias photodiode 28 preferably disposed in front of the iris 162 of the eye 6. The placement of at least one bias photodiode in this location allows the bias photodiode or photodiodes to be better exposed to light, compared to bias photodiodes, for example, disposed behind the iris. The bias photodiode 28 also contains the extended location of the ground return electrode 29, and the bias photodiode or photodiodes 28 provide additional voltage and/or current to the electrode stimulating unit 23 in the subretinal space. The bias photodiode or photodiodes 28 are electrically connected together in a series or parallel configuration to provide increased voltage and/or current as needed, and as is known in the art. For reference, other structures of the eye 6 that are shown are the cornea 158, lens 160, sclera 164, neuroretina 150, retinal pigment epithelium 152 and optic nerve 166, and the incident light images 156.

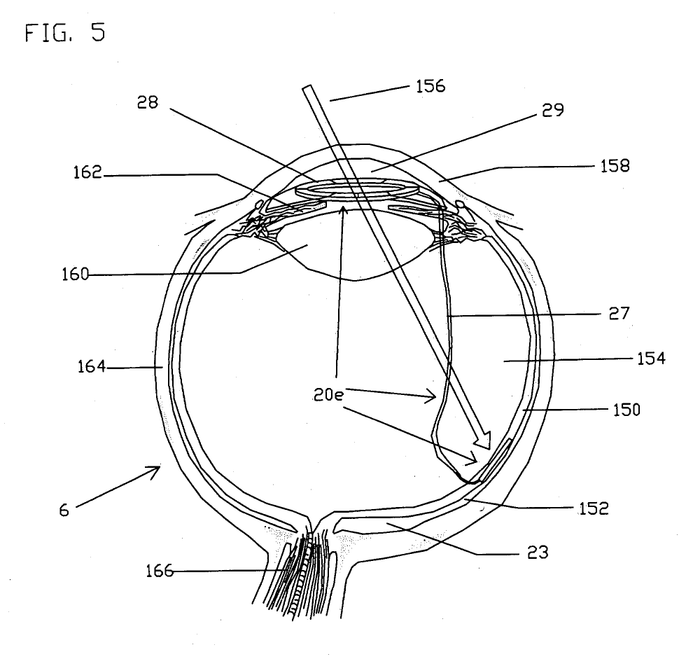

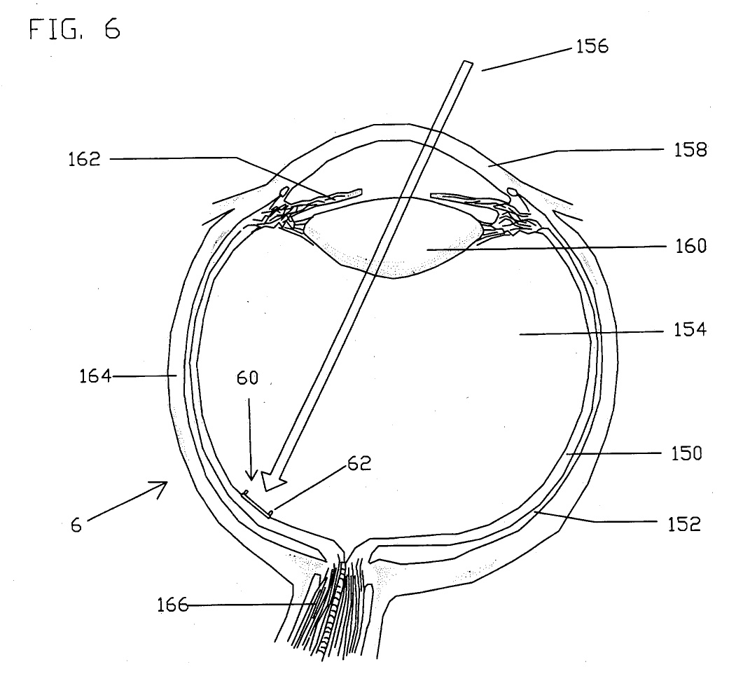

FIG. 6 is a cross-sectional view showing yet another RSD embodiment 60 implanted in an eye 6 on the epiretinal surface between the vitreous 154 and the neuroretina 150. This RSD embodiment 60 is similar to the RSD 10 of FIG. 3. However, in this embodiment, the RSD 60 is secured on the epiretinal surface by retinal tacks 62 or a biocompatible glue as is well known to those skilled in the art. Light 156 entering the eye 6 through the cornea 158 and lens 160 is focused onto the RSD 60. Electric current is generated by the RSD 60, which may comprise one or more photodiodes optionally connected in series or in reverse parallel fashion, to provide beneficial stimulation to the underlying neuroretina 150. Preferably the stimulation electrode that contacts the neuroretina is a cathode and the ground return electrode of the RSD 60 contacts the vitreous fluid 154 is the anode. However, the reversed position of the anode and the cathode is also suitable for electrical stimulation. For purposes of reference, other structures of the eye 6 that are shown are the iris 162, the sclera 164 and the optic nerve 166.

Indirect Stimulation

The embodiments described above relative to FIGS. 3-6 all possess the common characteristic that the electrical stimulus is provided directly to the neuroretina, i.e., there are substantially no intervening biological structures. In another aspect of the present invention, electrical stimulus may be applied to the neuroretina in an indirect fashion, i.e., via one or more intervening biological structures.

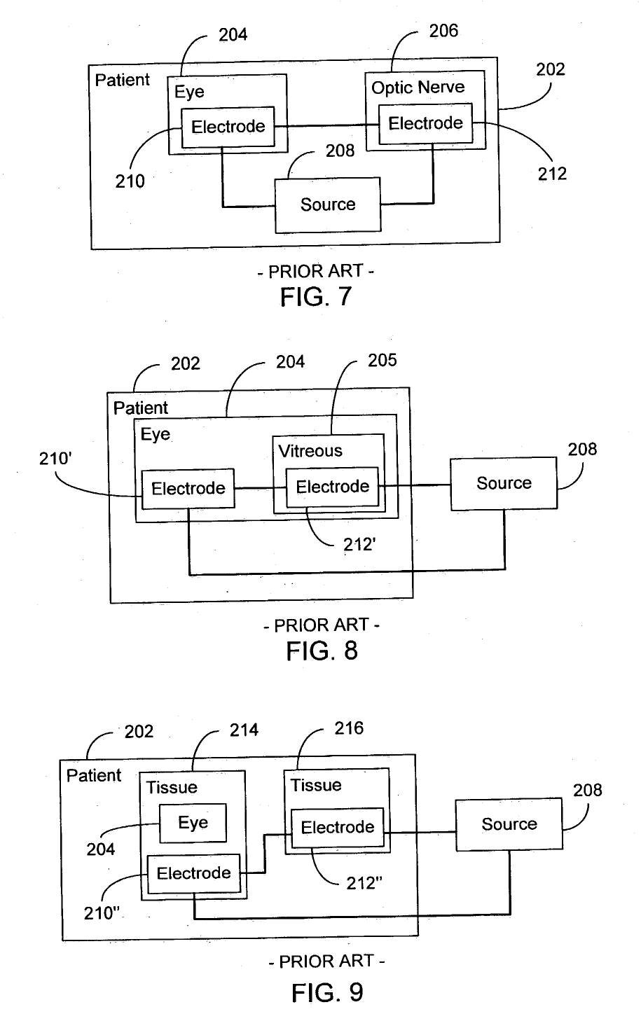

Various methods for indirect stimulation, as that term is defined herein, are known. FIGS. 7-9 are schematic illustrations of such prior art techniques. FIG. 7 illustrates a technique (described in U.S. Pat. No. 5,147,284 issued to Fedorov et al.; hereinafter “Fedorov”) in which electrical stimulation is applied to an eye 204 of a patient 202 via a pair of surgically implanted electrodes 210, 212 applied to surfaces of the eye 204 and optic nerve 206. A source of electrical stimulation 208 is provided coupled to the pair of electrodes 210, 212. In practice, the source 208 comprises an induction coil that provides electrical currents as a result of magnetic fields applied to the temporal region of the patient 202. While Fedorov reports improved vision in patients, the circumstances under which the patients were treated are not known and do not appear to have been subjected to peer review. Moreover, it will be readily evident to those having ordinary skill in the art that the implantation of an electrode in close proximity to the optic nerve 206 requires highly invasive and complicated surgery.

FIG. 8 illustrates a more recent technique proposed by Chow in U.S. Pat. No. 6,427,087. In particular, an electrode 210′ is placed in contact with tissues of the eye 204, whereas another electrode 212′ is placed within the vitreous cavity 205 (see also the vitreous cavity 154 illustrated in FIG. 1). It is believed that the resulting trans-retinal stimulation resulting from this configuration will result in more efficient stimulation.

Yet another approach is illustrated in FIG. 9 in which the stimulating 210″ and return 212″ electrodes, rather than being in direct contact with the eye 204, are instead placed upon external tissues 214, 216. Examples of this approach (sometimes referred to as microcurrent stimulation), particularly for the purpose of treating degenerative retinal diseases such as macular degeneration and retinitis pigmentosa, are taught in U.S. Pat. No. 5,522,864 to Wallace et al. and U.S. Pat. Nos. 6,035,236 and 6,275,735 to Jarding et al. Typically, the stimulating electrode 210″ is coupled to external tissue in close proximity to the eye 204, e.g., the eyelid, and the return electrode 212″ is coupled to distal external tissues such as the occipital lobe or arm of the patient 202. While anecdotal evidence of efficacy has been sporadically reported, no controlled, peer reviewed studies on humans are known to have been performed and, furthermore, the American Academy of Ophthalmology's Task Force on Complementary Therapies concluded in September 2000 that “strong evidence has not been found to demonstrate the effectiveness of microcurrent stimulation treatment of [age-related macular degeneration] compared to standard therapies.”

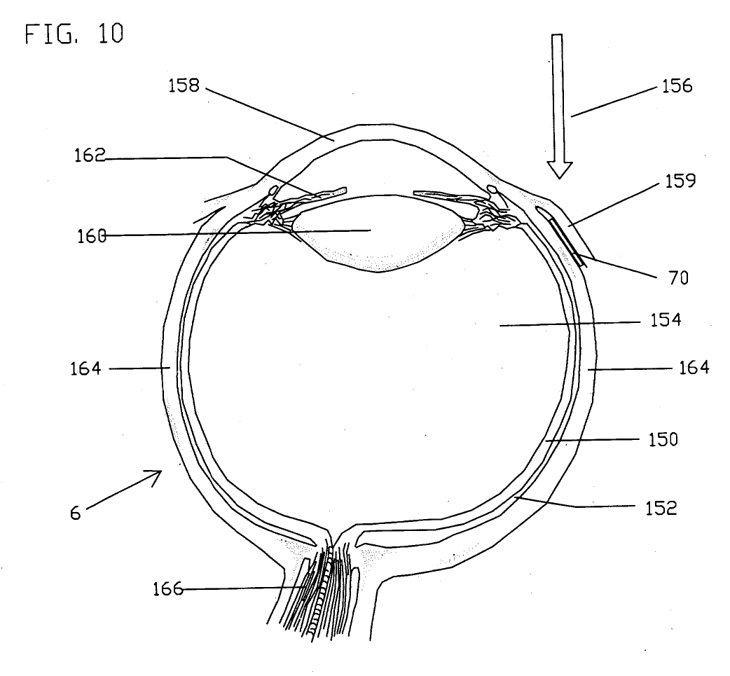

FIG. 10 illustrates a first technique for indirect stimulation in accordance with the present invention. In particular, FIG. 10 is a cross-sectional view showing a third embodiment RSD 70 implanted in an eye 6 on the anterior scleral surface between the conjunctiva 159 and the sclera 164 preferably nasal or temporal to the cornea. This RSD embodiment 70 is similar to the RSD 10 of FIG. 6. However, in this RSD embodiment 70, the RSD is secured in the subconjunctival space by the conjunctiva 159 on the RSD 70 anterior surface and the sclera 164 on the RSD 70 posterior surface. Light 156 passing through the conjunctiva 159 illuminates the RSD 70. Electric potential is generated by the RSD 70 that provides beneficial stimulation to the neuroretina 150 via conduction through the sclera 164. It is preferred that the stimulation electrode that contacts the sclera 164 is a cathode and the ground return electrode of the RSD 70 that contacts the conjunctiva 159 is the anode. However, the reversed position of the anode and the cathode is also suitable for electrical stimulation. For purposes of reference, other structures of the eye 6 that are shown are the iris 162, the sclera 164 and the optic nerve 166.

In addition to the preferred embodiments of the RSD described above, the devices in Table A are also preferred.

TABLE A

| Device | References |
|---|---|
| Artificial Silicon Retina (ASR ™) | (Chow, U.S. Pat. No. 5,016,633, |
| | 1991; Chow, U.S. Pat. No. |
| | 5,024,223, 1991) |
| Independent Surface Electrode | (Chow and Chow, U.S. Pat. No. |
| Microphotodiodes (ISEMCP) | 5,397,350, 1995; Chow and Chow, |
| | U.S. Pat. No. 5,556,423, 1996) |
| Independent Surface Electrode | (Chow and Chow, U.S. Pat. No. |
| Microphotodiodes with an electrical | 5,397,350, 1995; Chow and Chow, |
| capacitor (ISEMCP-Cs) | U.S. Pat. No. 5,556,423, 1996) |
| Multi-phasic Photodiode Retinal | (Chow and Chow, U.S. Pat. No. |
| Implants (MMRIs, such as MMRI-4) | 5,895,415, 1999; Chow and Chow, |
| | U.S. Pat. No. 6,230,057 B1, 2001) |
| Variable Gain Multi-phasic | (Chow and Chow, U.S. Pat. No. |
| Photodiode Retinal Implants | 6,389317, 2002) |
| (VGMMRIs) | |

Location of Electrical Stimuli

The electrical stimulation, if provided by implants such as the RSDs described above, may be provided subretinally, epiretinally, subsclerally (between the sclera and choroid), on the scleral surface, on the conjuctival surface and/or from or within any structure of the eye. Other means of providing electrical simulation to the retina and eye may include devices that deliver stimulation from the underside of the eyelid(s). Preferably, stimulation is from the subretinal space. Electrical stimulation from the exterior of the eyelid is not preferred.

Growth Factors

In addition to the endogenous retinal growth factors that are produced and released by electrical stimulation of retina cells by the methods of the invention described above, growth factors can also be instilled into the eye that further enhance retinal rescue and retina functional improvement. This additional step is attractive because Injecting growth factors, especially neurotrophic-type growth factors, have been reported to improve retinal function and provide limited neuronal rescue in eyes with retinal degeneration and dysfunction. These growth factors include, but are not limited to, glial cell line-derived neurotrophic factor (GDNF), nerve growth factor (NGF), brain derived neurotrophic growth factor (BDNGF), neurotropin-3 (NT-3), neurotropin-4 (NT-4), neurotropin-5 (NT-5), ciliary neurotropic factor (CNTF) and fibroblastic growth factor (FGF). These growth factors can be delivered to the eye by coating the RSD with growth factor(s) before implantation, by injection of the growth factor(s) into the locations of the subretinal space, vitreous cavity, subconjunctival space, subscleral space, and/or the anterior chamber either singly or in combination with each other, as a single dose or as multiple repeat doses before, during and/or after implantation of the RSD(s) or other electrical stimulating device.

Amplitude, Pattern and Frequency of Stimulation

Using the preferred RSDs, electrical stimulation is generated upon exposure to visible and/or infrared light (400 to greater than 750 nm); in the case of MMRIs, the NIP configuration provides a current when illuminated with visible light (400-750 nm), while the PIN configuration provides a current when illuminated with infrared light (greater than 750 nm). The RSDs, however, may be designed to respond to any wavelength or wavelength portions of ultraviolet, visible and/or infrared light, using methods and designs such as those described (Chow and Chow, U.S. Pat. No. 6,230,057 B1, 2001) and to produce any temporal pattern of stimulation. For example, the produced current per RSD may be 0.01 nA to 2,000,000 nA; most preferably 1 to 5000 nA and the temporal pattern of stimulation may be monophasic, biphasic or complex combinations of monophasic and biphasic waveforms with varying ramps of increasing and decreasing current and voltage. Electrical stimulation may also be provided continuously or intermittently. The electric current output of the RSD will depend on the degree of RSD stimulation by the appropriate light wavelengths or wavelength portions of light. The voltage potential of the RSD output is −20V to +20V, preferably −5V to +5V, and most preferably −1V to +1V.

Alternative Indirect Stimulation Embodiments

In addition to the indirect stimulation technique described above, the present invention also encompasses indirect stimulation techniques based on application of one or more electrodes to surface structures of the eye, as opposed to peripheral structures such as the optic nerve or eyelids. As used herein, surface structures of the eye may be divided into two classes, internal surface structures and external surface structures as described in greater detail below. In general, surface structures of the eye may be defined as any of several laminae (beginning most interiorly with the sclera in the case of internal surface structures) and forming or surrounding the eye, depending upon the specific region of the eye under consideration.

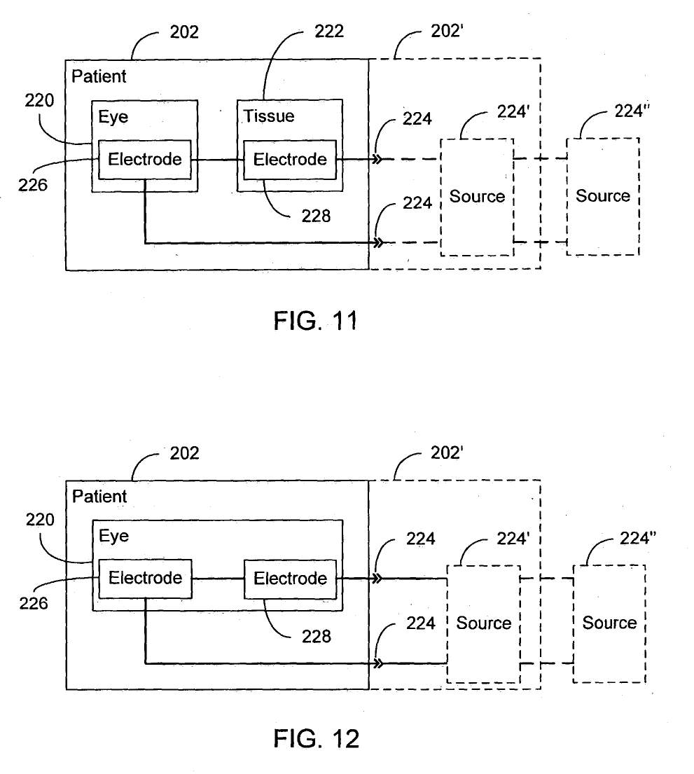

A schematic illustration of a second embodiment of indirect stimulation in accordance with the present invention is presented in FIG. 11. In this embodiment, at least one active or stimulating electrode 226 is applied to a surface structure of an eye 220. The at least one active electrode 226 is configured for chronic contact with the surface structure of the eye 220. As used herein, the term chronic encompasses not only continuous periods of time but also repetitive and/or periodic intervals of time. For example, the at least one active electrode 226 may be substantially permanently attached or otherwise coupled to the surface structure, or it may be configured to allow for repetitive placement in contact with, and subsequent removal from, the surface structure over a period of time established by a course of treatment. At least one return or ground electrode 228 is configured for application to tissues 222 substantially distant from the eyeball 220, e.g., outside the orbit of the eye. For example, in this embodiment, the at least one return electrode 228 may be coupled to the temporal or occipital regions of the patient or, more distally, to the neck, shoulder, chest, arms or legs of the patient. Additionally, the at least one return electrode 228 may be configured for chronic or temporary application to the tissue 222. For example, the at least one return electrode 228 may comprise one or more implantable electrodes substantially permanently coupled to the tissue 222 or it may comprise one or more temporary cutaneous electrodes secured with an adhesive and electrically coupled using a suitable conductive gel. Positioned in this manner, and given the relatively low resistance of the vitreous relative to the surface structures and surrounding tissues of the eye, the active and ground return electrodes establish a trans-retinal circuit such that application of an electrical stimulation signal to the active electrode will result in beneficial trans-retinal currents.

In addition to the electrodes 226, 228, the system illustrated in FIG. 11 also comprises a source of the electrical stimulation signal. The particular configuration of the source depends on whether the source is implemented entirely internally or externally, or combined internally and externally, relative to the patient. For example, in the case where only the active electrode 226 is configured to removably contact external surface structures of the eye and the return electrode 228 is configured for temporary cutaneous contact, the source may comprise one or more input terminals 224 for application of the electrical stimulation signal to the electrodes. In this case, the electrical stimulation signal is provided by an extraocular signal source 224′.

Alternatively, the source 224′ may be entirely internal to the patient 202′ as in the case of an implantable battery and, optionally, signal generation circuitry (not shown). In this case, it is assumed that the at least one return electrode 228 is likewise chronically implanted in the patient 202′, thereby vitiating the need for any input terminals 224.

Further still, the source may be implemented as a combination of internal 224′ and external 224″ (relative to the patient) components. For example, the internal source component 224′ may comprise a receiver induction coil implanted subcutaneously and the external source component 224″ may comprise transmitting coil that may be precisely aligned with the receiver induction coil. As know in the art, such transmitter/receiver coil pairs may be used to transmit power and data that may be used to provide the electrical stimulation signal.

In practice, the electrical stimulation signal provided by the source may comprise virtually any type of waveform demonstrating a beneficial effect. For example, the electrical stimulation signal may comprise an anodic or cathodic direct current signal or a time-varying waveform such as a square, sine, triangular, saw tooth signal or any other similar waveform. Preferably, the electrical stimulation signal comprises a bi-phasic waveform that is balanced in the sense a net zero charge is applied to the retina over a period of time. This may be achieved, by way of non-exhaustive examples, through the use of a signal comprising a continuous train of equal-duration bi-phasic pulses; equal-duration bi-phasic pulses separated by periods of quiescence; varying duration and amplitude bi-phasic, charge balanced pulses; combinations of the above; etc. Pulse frequencies may range anywhere from 10 KHz down to 0.001 Hz or, in the extreme, even a continuous monophasic waveform, i.e., 0 Hz. Those having ordinary skill in the art will appreciate that the particular type of electrical stimulation signal used is a matter of design choice and is selected so as to provide maximum beneficial effect.

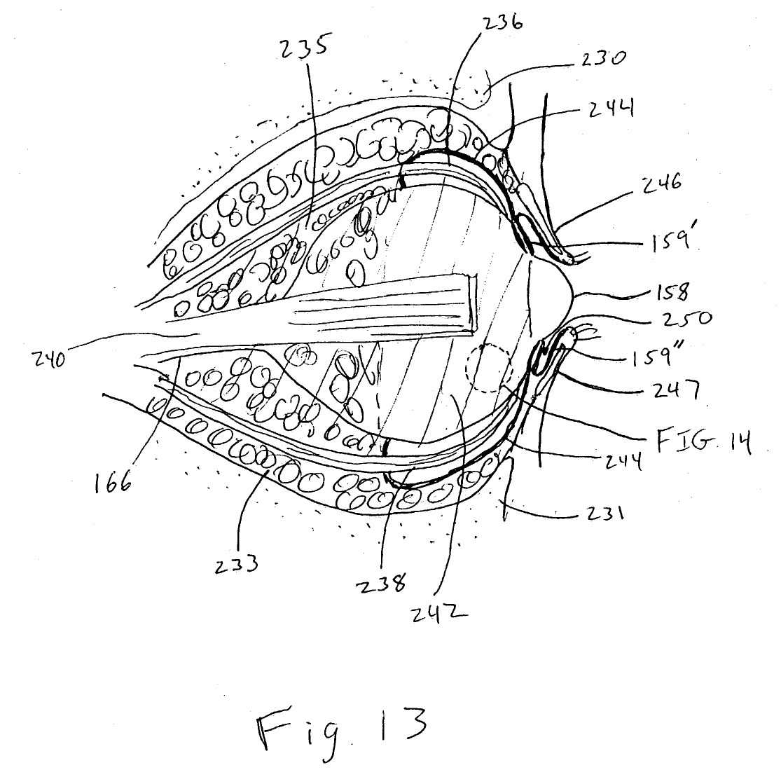

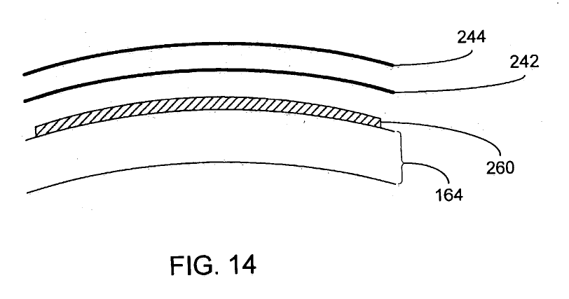

A schematic illustration of a third embodiment of indirect stimulation in accordance with the present invention is presented in FIG. 12. In this embodiment, the at least one active electrode 226 is applied to a first surface structure of the eye 220 and the at least one return electrode 228 is applied to a second surface structure of the eye 220. In practice, the first and second surface structures may be the same or different surface structures. The source 224, 224′, 224″ of the electrical stimulation signal in this embodiment may comprise any of the alternatives described above relative to FIG. 11. It is anticipated that the third embodiment of indirect stimulation illustrated in FIG. 12 will provide heightened stimulation of the retina given the relative proximity of electrodes to the retina. The various surface structures applicable to the present invention are further described below with reference to FIGS. 13 and 14.

Referring now to FIG. 13, an eye and surrounding structures are illustrated. The ocular orbit is defined by bone structures 230, 231. Within the orbit, a layer of extraconal fat 233 and intraconal fat 235 surround the eyeball. The fat layers 233, 235 are separated from each other by a cone defined by superior 236, inferior 238 and lateral 240 extraocular muscles as well as an intermuscular septum 242 connecting the muscles. The optic nerve 166 exits the orbit posteriorly, whereas the anterior portion of the eyeball is formed by a portion of the sclera and the cornea 158. The so-called Tenon's capsule 244 (partially shown) separates the eyeball from the orbital fat and forms a socket within which the eyeball moves. The upper and lower eyelids 246, 247 enclose and protect the anterior portion of the eyeball. The conjunctiva comprises the bulbar conjunctiva 159′ overlying the anterior portion of the sclera and the palpebral conjunctiva 159″ overlying the inner surface of the upper and lower eyelids 246, 247. The fold between the bulbar and palpebral conjunctiva 159′, 159″ gives rise to a conjunctival formix 250. In the context of the present invention, external surface structures comprise those surface structures that are accessible via the palpebral fissure defined by the eyelids, i.e., the cornea 158 and the conjunctiva 159. Internal surface structures are defined as those surface structures posterior to the bulbar conjunctiva 159′ and comprise the various laminae beginning with the sclera and its overlying structures, which overlying structures are dependent upon the particular region of the eyeball under consideration.

FIG. 14 schematically illustrates the various surface structures present at the exemplary region indicated in FIG. 13. The dimensions shown are not to scale. The sclera 164 forms the innermost surface structure. Moving outwardly from the sclera 164, the episclera 260 is a thin, loose layer of connective tissue forming the outer surface of the sclera 164. The intermuscular septum 242 resides above the episclera 260, and Tenon's capsule 244 resides above the intermuscular septum 242. Each of the layers illustrated in FIG. 14 comprises, for purposes of the instant invention, a separate surface structure to which an electrode may be applied. Those having ordinary skill in the art will appreciate that other regions of the eyeball may have surface structure layers different from those illustrated in FIG. 14.

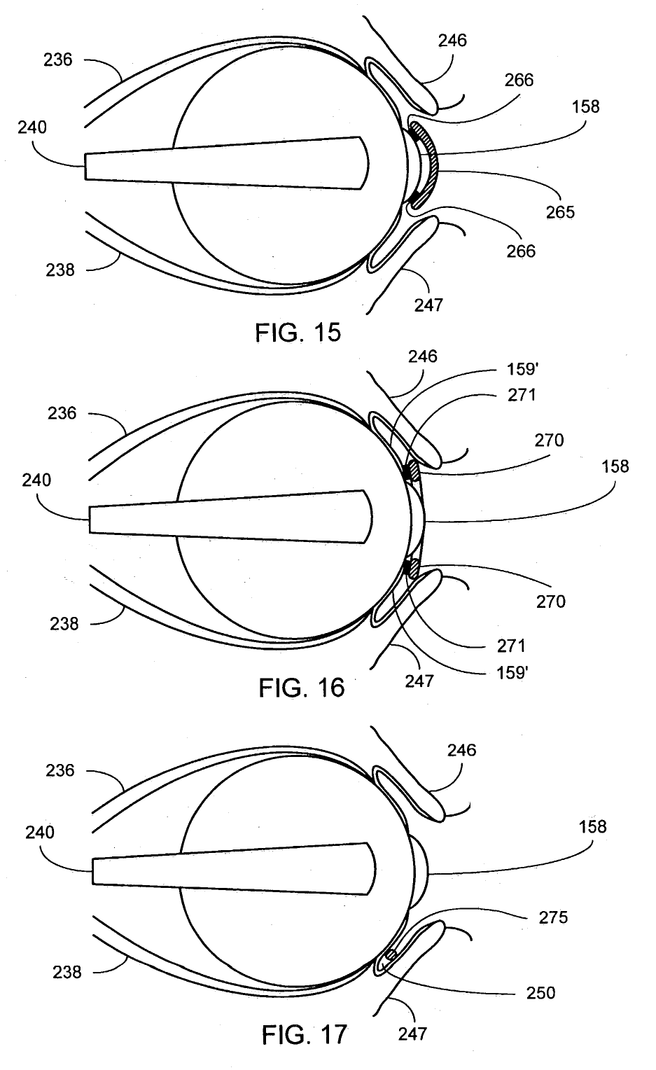

Various exemplary implementations of the second and third embodiments of FIGS. 11 and 12 are schematically illustrated with respect to FIGS. 15-19. For reference, each of FIGS. 15-17 illustrate the superior 236, inferior 238 and lateral 240 extraocular muscles, the upper and lower eyelids 246,247 and the cornea 158. FIG. 15 illustrates an embodiment in which a contact lens body 265 supports one or more corneal electrodes 266. Various materials for fabricating the supporting body 265 and the at least one corneal electrode 266 are known to those having ordinary skill in the art. Although it is preferred that the at least one corneal electrode 266 be employed as an active electrode, it may likewise be employed as a return electrode. (This is also true of the other embodiments illustrated in FIGS. 16 and 17.) The at least one corneal electrode 266 may comprise a plurality of discrete electrodes arranged, for example, in a ring formation affixed in proximity to the periphery of the supporting body 265, or may comprise a single annular electrode similar affixed to the supporting body 265. Alternatively, the at least one corneal electrode 266 may be arranged closer to the central region of the cornea. Where a plurality of electrodes 266 are employed, each electrode may be individually selectable, i.e., each of the electrodes is separately addressable and the electrical stimulation signal may be applied to each electrode individually. When the electrical stimulation signal is applied, current will flow through the cornea and vitreous cavity, across the retina and back to the return electrode. Note that, for ease of illustration, none of FIGS. 15-17 illustrate the complementary electrode, nor do FIGS. 15-19 illustrate the electrical connections between the electrodes and the source of the electrical stimulation signal, which connections will be readily devisable as a matter of design choice by those having ordinary skill in the art.

FIG. 16 illustrates another embodiment in which an annular supporting body 270 provides support for at least one epi-conjunctival electrode 271. Once again, various materials for fabricating the supporting body 270 and the at least one epi-conjunctival electrode 271 are known to those having ordinary skill in the art. As in the embodiment of FIG. 15, the at least one epi-conjunctival electrode 271 may comprise a plurality of individually selectable electrodes or a single annular electrode as a matter of design choice. In the example shown in FIG. 16, the at least one epi-conjunctival electrode 271 contacts the bulbar conjunctiva 159′ in close proximity to the cornea 158. However, additionally or alternatively, the at least one epi-conjunctival electrode 271 may be placed more distally from the cornea 158 and yet still in contact with the bulbar conjunctiva 159′. Regardless, current flow will traverse the bulbar conjunctiva 159′, the sclera, vitreous cavity and retina.

FIG. 17 illustrates yet another embodiment in which an electrode 275 is placed in epi-conjunctival contact within the conjunctival formix 250. In practice, the electrode 275 may comprise a fibrous or filamentary electrode such as a “DTL” electrode. DTL electrodes are particularly advantageous because they are known to be well tolerated by patients given their relatively slender dimensions. Although a single electrode is illustrated in the lower conjunctival formix 250, an electrode may also be placed in the upper conjunctival formix as an alternative, or in addition to, the lower electrode. Furthermore, more than one electrode can be placed into either of the formices at a single time. As in the embodiment of FIG. 16, current flow in the embodiment of FIG. 17 will traverse the bulbar conjunctiva 159′, the sclera, vitreous cavity and retina.

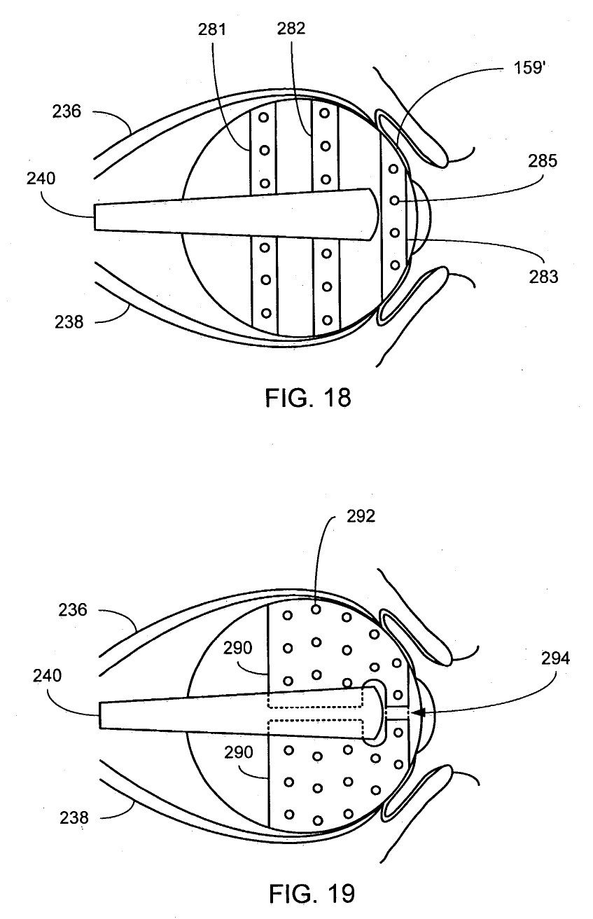

Each of FIGS. 15-17 illustrates embodiments in which electrodes are placed in contact with external surface structures of the eye. FIG. 18 schematically illustrates an embodiment in which electrodes are applied to internal surface structures. In particular, one or more supporting rings 281-283 are implanted in contact with internal surface structures. Note that although the third ring 283 is placed in contact with a substantially anterior portion of the eye, it is implanted beneath the bulbar conjunctiva 159′. However, a hybrid internal/external surface structure technique may be possible if the third ring 283 were placed above the bulbar conjunctiva 159′ in a manner similar to that illustrated in FIG. 16. Techniques for introducing such rings into the orbit and for securing them to the eye are know in the art, particularly from the use of so-called scleral buckles. For example, each ring may be sutured in place in accordance with such techniques. Note that the first and second rings 281, 282 are preferably placed beneath the muscles 236, 238, 240 in accordance with known techniques.

Each ring comprises at least one electrode 285 and, in a preferred embodiment, each ring comprises a plurality of electrodes. Suitable materials for fabricating the supporting rings and electrodes are known to those having ordinary skill in the art. Preferably, each electrode is individually selectable. Additionally, each individual electrode may be electrically configured to act as an active electrode or a return electrode. In this manner, each ring 281-283 may comprise both active and return electrodes. In such an embodiment, it may be preferable to interleave active and return electrodes and, further, to antipodally arrange the active and return electrodes. An antipodal arrangement of electrodes will give rise to a trans-retinal current path that is substantially perpendicular to the retinal surface. Additionally, being individually selectable, each electrode in an antipodal electrode pair could be periodically switched between active and return operation. Further still, electrodes between rings could be activated as stimulating pairs, e.g., an electrode from a first ring 281 could be operated as an active electrode and an electrode from a second ring 282 could be operated as a return electrode, and vice versa. Although a specific number of supporting rings 281-283 positioned in substantially vertical orientations are illustrated in FIG. 18, it is understood that a greater or lesser number of such rings could be employed and, further, that the orientation of such rings need not be limited to substantially vertical. Taken to an extreme, the supporting rings 281-283 could be eliminated and, instead, each electrode 285 may comprise a separate, independent supporting member such that individual electrodes may be implanted at specific locations on specific internal surface structures.

Yet another embodiment providing contact with internal surface structures is illustrated in FIG. 19. In this embodiment, one or more supporting sheaths 290 comprising a plurality of electrodes 292 are positioned and secured in contact with internal surface structures of the eye. The discussion above with regard to individually selectable and antipodal electrodes relative to FIG. 18 equally applies to the arrangement of FIG. 19. To accommodate the presence of various ocular structures, such as the connections between the various muscles 236, 238, 240 and the sclera, openings 294 may be provided. In the example illustrated in FIG. 19, a plurality of sheaths 290 are provided such that each sheath 290 covers the surfaces between adjacent muscles with the openings 294 thereby being formed by the adjacency of the sheaths 290 when implanted. In the case where a single sheath 290 surrounds at least one of the muscles, the anterior portion of the opening 294 could be fabricated such that a unitary body is provided (illustrated with dotted lines) whereas the opening of the posterior portion of the opening 294 would allow flexing of the sheath 290 for placement underneath the muscle. Further still, rather than trying to maneuver the sheaths 290 around the muscles, holes could be provided within the otherwise continuous sheaths 290. In this case, the muscles would need to be severed first to allow positioning of the sheaths, followed by reattachment of the muscles at positions corresponding to the holes. Regardless of the particular configuration, the embodiment illustrated in FIG. 19 allows multiple electrodes to be placed in contact with internal surface structures of the eye to facilitate indirect stimulation of the retina.

Equivalents

Although particular embodiments have been disclosed herein in detail, this has been done for purposes of illustration only and is not intended to be limiting with respect to the scope of the appended claims that follow. In particular, it is contemplated by the inventors that various substitutions, alterations, and modifications may be made to the invention without departing from the spirit and scope of the invention as defined by the claims. Other aspects, advantages, and modifications are considered to be within the scope of the following claims.

We claim:

1. A device for preventive or therapeutic treatment of degenerative retinal disease comprising:
a source of an electrical stimulation signal;
at least one stimulating electrode, coupled to the source, configured for chronic contact with a surface structure of an eyeball; and
at least one return electrode, coupled to the source, configured for contact with conductive biological tissue substantially distant from the eyeball,
wherein the electrical stimulation signal is applied to the eyeball via the at least one stimulating electrode and the at least one return electrode.

2. The device of claim 1, wherein the source comprises a battery.

3. The device of claim 1, wherein the source comprises an induction coil.

4. The device of claim 1, wherein the source comprises a connection to an extraocular signal source.

5. The device of claim 1, wherein the at least one stimulating electrode is configured for corneal contact.

6. The device of claim 1, wherein the at least one stimulating electrode is configured for epi-conjunctival contact.

7. The device of claim 1, wherein the at least one stimulating electrode comprises a plurality of stimulating electrodes arranged in at least one ring formation.

8. The device of claim 7, wherein the at least one ring formation is configured for application to an external surface structure of the eyeball.

9. The device of claim 7, wherein the at least one ring formation is configured for application to an internal surface structure of the eyeball.

10. The device of claim 1, wherein the at least one return electrode is configured for chronic contact with the conductive biological tissue.

11. A device for preventive or therapeutic treatment of degenerative retinal disease comprising:
a source of an electrical stimulation signal;
at least one stimulating electrode, coupled to the source, configured for chronic contact with a first surface structure of an eyeball; and
at least one return electrode, coupled to the source, configured for chronic contact with a second surface structure of the eyeball
wherein the electrical stimulation signal is applied to the eyeball via the at least one stimulating electrode and the at least one return electrode.

12. The device of claim 11, wherein the source comprises a battery.

13. The device of claim 11, wherein the source comprises an induction coil.

14. The device of claim 11, wherein the source comprises a connection to an extraocular signal source.

15. The device of claim 11, wherein the at least one stimulating electrode is configured for application to an external surface structure of the eyeball.

16. The device of claim 11, wherein the at least one stimulating electrode is configured for application to an internal surface structure of the eyeball.

17. The device of claim 11, wherein the at least one return electrode is configured for application to an external surface structure of the eyeball.

18. The device of claim 11, wherein the at least one return electrode is configured for application to an internal surface structure of the eyeball.

19. The device of claim 11, wherein the at least one stimulating electrode comprises a plurality of stimulating electrodes arranged in a ring formation.

20. The device of claim 11, wherein the at least one stimulating electrode and the at least one return electrode are arranged in at least one ring formation.

21. The device of claim 20, wherein each of the at least one stimulating electrode occupies a substantially antipodal position in the at least one ring formation relative to a corresponding one of the at least one return electrode.

22. The device of claim 20, wherein the at least one stimulating electrode and the at least one return electrode are interleaved in the at least one ring formation.

23. A method for preventive or therapeutic treatment of degenerative retinal disease, the method comprising:
chronically applying at least one stimulating electrode to a first surface structure of an eyeball;
applying at least one return electrode to conductive biological tissue substantially distant from the eyeball; and
applying an electrical stimulation signal to the eyeball via the at least one stimulating electrode and the at least one return electrode.

24. The method of claim 23, wherein chronically applying the at least one stimulating electrode further comprises chronically applying the at least one stimulating electrode to an external surface structure of the eyeball.

25. The method of claim 23, wherein chronically applying the at least one stimulating electrode further comprises chronically applying the at least one stimulating electrode to an internal surface structure of the eyeball.

26. The method of claim 23, wherein applying the at least one return electrode comprises chronically applying the at least one return electrode to the conductive biological tissue.

27. A method for preventive or therapeutic treatment of degenerative retinal disease, the method comprising:
chronically applying at least one stimulating electrode to a first surface structure of an eyeball;
chronically applying at least one return electrode to a second surface structure of the eyeball; and
applying an electrical stimulation signal to the eyeball via the at least one stimulating electrode and the at least one return electrode.

28. The method of claim 27, wherein chronically applying the at least one stimulating electrode further comprises chronically applying the at least one stimulating electrode to an external surface structure of the eyeball.

29. The method of claim 27, wherein chronically applying the at least one stimulating electrode further comprises chronically applying the at least one stimulating electrode to an internal surface structure of the eyeball.

30. The method of claim 27, wherein chronically applying the at least one return electrode further comprises chronically applying the at least one return electrode to an external surface structure of the eyeball.

31. The method of claim 27, wherein chronically applying the at least one return electrode further comprises chronically applying the at least one return electrode to an internal surface structure of the eyeball.

32. The device of claim 27, wherein chronically applying the at least one stimulating electrode comprises chronically applying a plurality of stimulating electrodes arranged in at least one ring formation.

33. The method of claim 32, wherein chronically applying the at least one return electrode further comprises chronically applying the at least one return electrode within the at least one ring formation.

34. The method of claim 33, wherein chronically applying the at least one stimulating electrode and the at least one return electrode further comprises chronically applying each of the at least one stimulating electrode at a substantially antipodal position in the at least one ring formation relative to a corresponding one of the at least one return electrode.

35. The method of claim 33, wherein chronically applying the at least one stimulating electrode and the at least one return electrode further comprises interleaving the at least one stimulating electrode and the at least one return electrode in the at least one ring formation.

36. An implantable device for use in preventive or therapeutic treatment of degenerative retinal disease comprising:
a body member configured for chronic contact with an internal surface structure of an eyeball;
at least one stimulating electrode maintained in a substantially fixed position by the body member; and
at least one return electrode maintained in a substantially fixed position by the body member.

37. The implantable device of claim 36, wherein the body member subtends an angle comprising at least a portion of a circumference of the eyeball.

38. The implantable device of claim 37, wherein the at least one stimulating electrode and the at least one return electrode are arranged in at least one ring formation.

39. The implantable device of claim 38, wherein each of the at least one stimulating electrode occupies a substantially antipodal position in the at least one ring formation relative to a corresponding one of the at least one return electrode.
